(19) United States
(12) Patent Application Publication
(10) Pub. No.: US 2017/0270280 A1
(43) Pub. Date: Sep. 21, 2017

(54) PROCESS FOR DETERMINING AND TRACKING THE APTITUDE OF MEDICAL RESIDENTS

(71) Applicant: Justin Massengale, Brookline Village, MA (US)

(72) Inventor: Justin Massengale, Brookline Village, MA (US)

(21) Appl. No.: 15/462,024

(22) Filed: Mar. 17, 2017

Related U.S. Application Data
(60) Provisional application No. 62/309,529, filed on Mar. 17, 2016.

(51) Int. Cl. G06F 19/00; G09B 19/00; G09B 7/06

(52) U.S. Cl. CPC G06F 19/363; G09B 7/06; G09B 19/00; G06F 19/327

(57) ABSTRACT

A process of evaluating the residential clinical performance of a medical resident based upon feedback from evaluators includes generating a number of questionnaires. The questionnaires contain questions related to competency area milestones for the medical resident. E-mails are sent to the medical resident with access codes for the questionnaires. The medical resident then works with an evaluator to treat a patient, the patient receiving healthcare related to a competency area milestone. The medical resident then sends the evaluator an access code for a questionnaire related to the competency area milestone. The evaluator uses the access code to access the questionnaire and answers the questions on the questionnaire. Answers are logged in a database.

### CROSS REFERENCE TO RELATED APPLICATION

This application is a U.S. non-provisional patent application which claims priority from U.S. Provisional Application for Patent No. 62/309,529 filed Mar. 17, 2016 which is incorporated herein by reference.

### FIELD OF THE INVENTION

The subject disclosure relates to aptitude evaluation, and more particularly to improved processes of evaluating medical residents.

### BACKGROUND OF THE INVENTION

For recent medical school graduates, completing a medical residency is often a requirement to obtain a license to practice medical in a given area. Over the course of a medical residency program, the medical works with faculty evaluators to treat patients in a number of specific healthcare areas. At the end of each year of medical residency, the medical resident is required to proficient in a number of competency area milestones related to specific healthcare areas.

At the end of a semi-annular period, a review is conducted where the medical resident's performance is rated by the evaluators they worked with during that period. During this period, the residents are graded on their aptitude related to the specific competency area milestones pertinent to them, which depends upon which year of their residency they are completing. Unfortunately, the evaluators may have worked with the resident at any time during the period six month period. Therefore the accuracy of the evaluations can suffer from inaccuracies that are born from the evaluators needing to recall events that took place many months ago.

Further, since the review is based solely on what the evaluator describes with regards to the resident, this language can be difficult to translate into objective criteria to assess the overall performance resident. This leads to additional problems in generating an overall report on the performance of the resident. Generating an overall report based on the reporting of each evaluator can be time consuming and can result in a report that has different amounts of detail or has different areas of focus depending on the evaluator.

### SUMMARY OF THE INVENTION

The subject disclosure overcomes the drawbacks of the prior art, improving efficiency and accuracy, by providing a process that tracks the aptitude of medical residents using a uniform system to streamline the evaluation process while receiving and storing feedback in real time.

In some embodiments, the subject technology relates to a process of evaluating the residential clinical performance of a medical resident based upon feedback from a plurality of evaluators. First, a plurality of questionnaires are generated. Each questionnaire relates to one of a plurality of competency area milestones. In some cases, the competency area milestones are related to healthcare treatment areas in which the medical resident is being evaluated. The healthcare treatment areas can depend on the years of medical residency completed by the medical resident. The questionnaires also have a plurality of questions related to the one of the competency area milestones. A plurality of e-mails are sent to the medical resident, each e-mail containing an access code which provides access to one of the questionnaires. The medical resident works with one of the evaluators on a patient receiving healthcare related to one of the competency area milestones. The medical resident then sends, to the one of the evaluators, one of the access codes providing access to a questionnaires that relates to the competency area milestone relevant to the healthcare treatment just provided by the medical resident and evaluator. The evaluator then uses the access code to access the questionnaire that relates to that competency area milestone. The evaluator then provides answers to one or more of the questions on the questionnaire that was accessed and the answers are logged in an evaluation database. In one embodiment, the evaluator provides answers by opening an application on their mobile device and entering the access code. In some embodiments, answers are provided by selecting one of five options. The options can be the following: proficient; competent; early learner; unsatisfactory; and pass. In some cases, the steps of treating the patient to logging the answers can be repeated until an answer option of “proficient” is selected at least once as an option in response to each questionnaire related to each competency area milestone, at which time the resident can be notified. Throughout this process, the answers can be reviewed to track the progress of the medical resident towards their competency area milestone.

In some embodiments, the steps of treating the patient to logging the answers can be repeated throughout a period of time (i.e. an evaluation period). A determination can then be made regarding the total progress made by the medical resident towards competency area milestones during the period of time (i.e. evaluation period), the total progress being based upon the answers provided by the evaluators. An overall evaluation report can then be created based upon the answers provided.

In some embodiments, the process involves generating questionnaires as described above, however, access codes are not provided to the medical resident. The medical resident then works with an evaluator to provide healthcare services to a patient that are related to one of the competency area milestones. The evaluator is then provided with one of the questionnaires related to the competency area milestone associated with the healthcare provided to the patient. The evaluator then provides a plurality of answers to questions on the questionnaire. The answers can be provided, in some instances, by selecting one of five options. The options may include the following: proficient; competent; early learner; unsatisfactory; and pass. The answers are logged in an evaluation database. In some embodiments, the progress of the medical resident towards their competency area milestones can be determined based on the answers provided.

In some embodiments, the steps of treating the patient through logging the answers in an evaluation database can be repeated, as the medical resident treats other patients, throughout an evaluation period. The total progress made by the medical resident towards competency area milestones during the evaluation period can be determined based upon the answers provided by the evaluators. An overall evaluation report can also be created based upon the answers provided.

### BRIEF DESCRIPTION OF THE DRAWINGS

So that those having ordinary skill in the art to which the disclosed system pertains will more readily understand how to make and use the same, reference may be had to the following drawings.

FIG. 1 is a block diagram showing some of the parts of a system in accordance with the subject technology.

FIG. 2 is a flowchart showing a process in accordance with the subject technology.

### DETAILED DESCRIPTION OF PREFERRED EMBODIMENT

The subject technology overcomes many of the prior art problems associated with the evaluation of medical residents. The advantages, and other features of the systems and methods disclosed herein, will become more readily apparent to those having ordinary skill in the art from the following detailed description of certain preferred embodiments taken in conjunction with the drawings which set forth representative embodiments of the present invention.

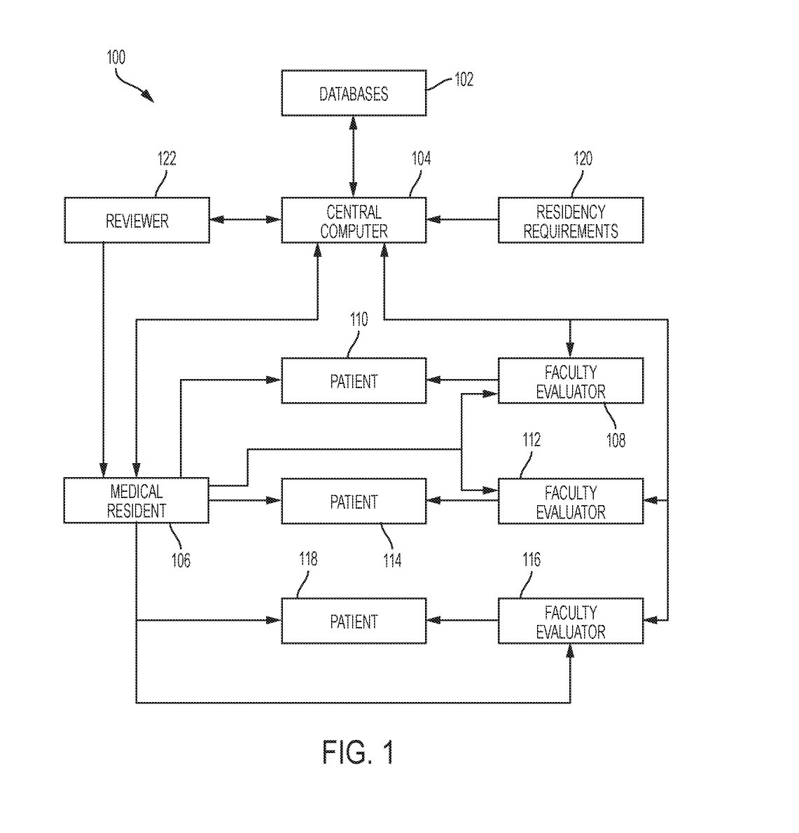

Referring now to FIG. 1 a block diagram showing some of the parts of a system in accordance with the subject technology is show generally at 100. The lines and arrows shown between the various parts are meant to show the interaction with, or the transfer of information between, those parts. The system 100 shows some of the components of the subject technology, but is not all inclusive. Rather, the system 100 is merely meant to help explain, from a general standpoint, what occurs as the subject technology is carried out. The system 100, and generally, the other processes described herein, are carried out as after a medical student graduates from medical school, and enters into a medical residency program, thereby becoming a medical resident. Throughout their time in a medical residency program, the medical resident will receive feedback from faculty evaluators regarding their performance in various healthcare areas which are associated with competency area milestones for their year-level of medical residency. Depending on the number of years of medical residency completed by the medical resident, passing grades for different competency area milestones are needed. The system 100 helps in receiving feedback on, tracking, logging, and generally evaluating the performance or aptitude of the medical resident with respect to those competency area milestones.

The system 100 is discussed with respect to various hardware and software components, such as databases 102 and a central computer 104. In various embodiments, a single database could be used rather than databases 102. Further, in some embodiments, the central computer 104 could be replaced by several computers, or by other devices functionally able to accomplish the tasks of the central computer. For example, application specific integrated circuits or dedicated hardware could be used to carry out various discrete functions of the central computer 104 described herein. Likewise, the databases 102 could be replaced by other components capable of storing information. The central computer 104 has access to one or more servers, and/or communicates with a distributed computer network via communication channels, whether wired or wireless, to transmit information between the central computer 104 and the other parts of the system 100. For example, in some embodiments, the central computer 104 has access to the internet. All of the hardware, computer, servers, databases, and devices may include particular user-features such as buttons, scanners and card readers, whether virtual or hard, that are specific to accomplish an aspect of the subject technology. Further, the database 102, the central computer 104, or their replacement parts, could operate autonomously or be controlled by one or more users.

Still referring to FIG. 1, the medical resident 106 and a faculty evaluator 108 work together to provide healthcare services to a patient 110. The healthcare services relate to one of the medical competency milestone requirements for the medical residency program. Therefore once the treatment is complete, the faculty evaluator 108 can provide an evaluation of the medical resident's 106 aptitude with respect to that medical competency milestone. To accomplish this, a questionnaire related to that medical competency milestone for evaluating the medical resident's 106 performance is provided to the faculty evaluator 108. The questionnaire can come from the database 102 and can be provided to the faculty evaluator by the central computer 104. Each questionnaire can be worded verbatim using the language of an individual competency milestone for a single level of training for the medical residency program. In some embodiments, the central computer 104 can provide the medical resident 106 with an access code which the medical resident 106 can provide to the faculty evaluator 108. The faculty evaluator 108 can then use the access code to access the questionnaire from the central computer 104. In some embodiments the access code is provided to the medical resident 106 through an e-mail which the medical resident 106 can then forward to the faculty evaluator 108. Each e-mail contains a single access code that allows the faculty evaluator 108 to gain access to the questionnaire for a single year level (i.e. a certain year of medical residency) of a single milestone for the resident 106. In some cases, these e-mails are generated automatically by the central computer 104 and sent to the resident 106 (and other residents) at the beginning of the medical residency program year. The faculty evaluator 108 can then open an application on their mobile device and enter the access code to complete the questionnaire. The central computer 104 can then compare the results of the questionnaire to a known medical residency requirements 120 for completing the medical residency program. The residency requirements 120 can be input into the central computer 104, or can reside elsewhere, such as in a database which the central computer 104 has access to.

This process can then be repeated between the medical resident 106 and additional faculty evaluators 112, 116 by treating additional patients 114, 118. If the healthcare treatment received by the additional patients 114, 118 is related to a medical competency milestone in which the medical resident 106 has not yet received a passing grade, the faculty evaluator 112, 116 can be asked to fill out a questionnaire, as discussed above. While three different faculty evaluators 108, 112, 116 are shown as well as three different patients 110, 114, 118, this is merely for illustrative purposes. Over the course of a medical residency program, a medical resident 106 will likely treat many more patients and work with many more faculty evaluators than shown herein. Further, the medical resident 106 might work with the same faculty advisor on numerous different patients, or work on one patient with several faculty advisors.

As the faculty evaluators 108, 112, 116 provide answers to the questionnaires to the central computer 104, the central computer 104 stores the answers in one or more databases 102. The central computer 104 can then recall the answers from the databases 102 as needed. For example, if at some point the answers to the questionnaires indicate that the medical resident 106 has received passing grades for every competency area milestone needed to complete their medical residency program, a notification can be provided to the medical resident 106. Similarly, at some point, a reviewer 122 may wish to review the progress of the medical resident 106. For example, it is sometimes beneficial to conduct a semiannual review. To streamline this review, the reviewer 122 can obtain a report, from the central computer 104, indicating every medical competency milestone required for the medical resident 106 per the residency requirements 120. The report obtained by the reviewer 122 can also show which of those medical competency milestones the medical resident 106 has achieved a passing grade in, and which ones the resident 106 must still achieve a passing grade in to complete the medical residency program. The resident's 106 goal is to reach the point, by the end of the training year, to have no blank questionnaires (and preferably, no below passing grades) remaining for any milestone category for that year.

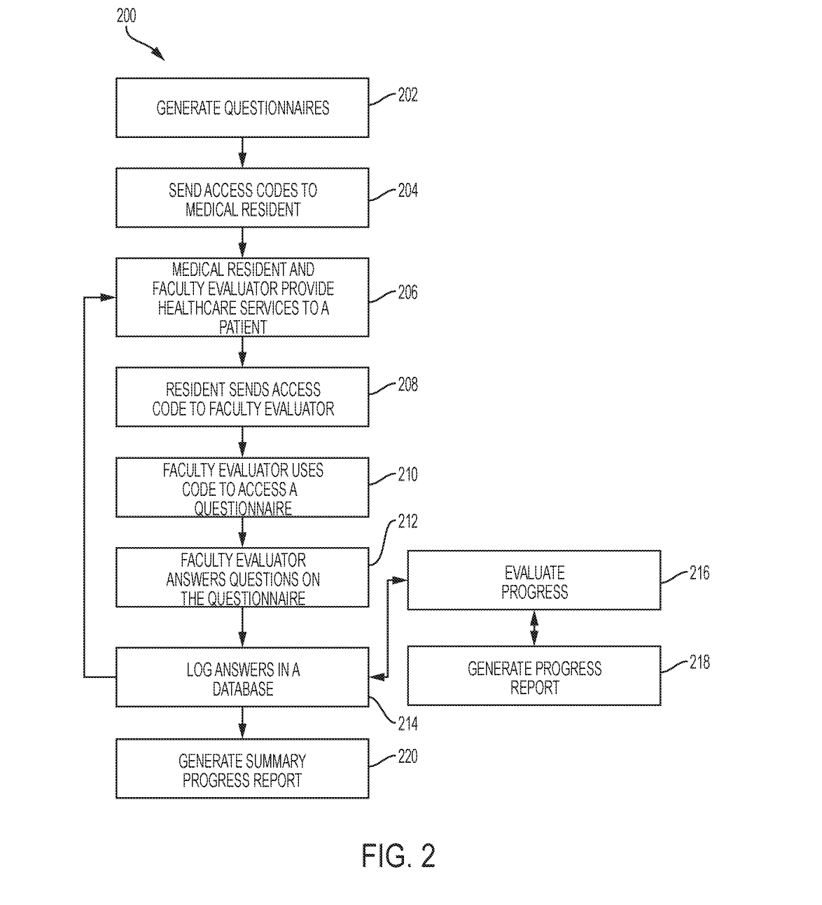

Referring now to FIG. 2, a flowchart of a process of evaluating the residential clinical performance of a medical resident based in accordance with the subject technology is shown generally at 200. Various components of the system 100, or other functionally similar systems, can be used to help carry out the process 200. For example, the process 200 may be practiced by a machine component that renders program code elements in a form that instructs a digital processing apparatus (e.g., computer or hardware device) to perform a sequence of functional steps similar to or corresponding to some of the steps shown in process 200. It should be noted that the process 200 merely represents several specific ways in which the subject technology can be carried out, and is not meant to be representative of all possible ways of implementing the subject technology.

The process 200 starts, at step 202, with the generation of a plurality of questionnaires. Each questionnaire is related to a competency area milestone and has a plurality of questions directed towards that competency area milestone. As discussed above, the competency area milestones are usually associated with a particular healthcare treatment area and relate to the requirements of a medical residency program. Therefore when a medical resident provides treatment in that particular healthcare treatment area to a patient, the performance of the medical resident with respect to the related competency area milestone can be assessed. In some embodiments the questionnaires are short, containing five or fewer questions, to precipitate an efficient evaluation process.

At step 204, access codes for accessing the questionnaires are provided to a medical resident. This can be accomplished by a central computer 104 sending a plurality of e-mails to the medical resident, each e-mail containing an access code which provides access to one questionnaire. Eventually, at step 206, the medical resident works with an evaluator to provide medical treatment to a patient, the medical treatment related to one of the competency area milestones. The term “evaluator” here is simply used to refer the role played by that person in evaluating the medical resident. It should be mentioned that the evaluators are often doctors, or other healthcare professions, who are licensed to provide healthcare services to patients. Thus, in many cases, the evaluator is a senior healthcare professional who, in addition to caring for the patient, must also supervise the medical resident to ensure the patient is receiving proper care.

After the patient has been treated, the medical resident sends one of the access codes received at step 204 to the evaluator at step 208. The access code allows the evaluator to access a questionnaire related to the competency area milestone that is related to the type of healthcare services the evaluator just worked with the resident to provide. For example, a resident in their first year who helps treat a patient with a brain tumor can forward the e-mail to the evaluator they worked with which contains the specific access code for the milestone category entitled “Brain Tumor Patient Care” for a first year resident. The evaluator will then use the access code to access and complete the questionnaire at steps 210 and 212. Preferably, steps 210 and 212 are carried out soon after the treatment takes place at step 206, while the treatment is still fresh in the mind of the evaluator. In some cases, step 210 can be accomplished by the evaluator simply entering the access code into an application on their mobile device. The pertinent questionnaire will then show up on the evaluator's mobile device so that they can answer the questions right on their device, thereby completing step 212. It should be noted that the evaluator need not use a mobile device to answer the questionnaire, and could instead answer the questionnaire, for example, by computer or by providing answers on a scantron or hand written form. The answers to the questions are then logged in a database at step 214 so they can later be retrieved when assessing the progress of the medical resident.

In some cases, the answers to the questionnaire can be streamlined, allowing the evaluator to select between a number of answer options. The uniformity of these answer options help track individual milestone “grades” for each resident over the course of the evaluation period, and also help generate summative data for performance reviews. Using uniform answer choices can remove some of the ambiguity with freeform evaluations, forcing the evaluator to select one of the specific choices for assessing the aptitude of the medical resident in the relevant area. Since the wording of the milestones is inseparable from the eventual performance “grades” the resident will receive in later semi-annual and summative reviews, using uniform answers for the questionnaires is inherently reflective of the milestone achievement for the performance periods to which they pertain. For example, in some cases, the five options for answer choices might be as follows: “proficient”; “competent”; “early learner”; “unsatisfactory”; and “pass.” “Pass” can be selected if, for whatever reason, the evaluator is unable to provide an informed evaluation in response to the call of that particular question. Otherwise, one of the other answer choices can be selected to describe the performance of the medical resident in that particular area.

Steps 206-214 can also be repeated by a single resident for a number of different patients to fill out the medical residency program requirements of the medical resident. The medical resident works with evaluators (sometimes new evaluators and sometimes the same ones) to provide treatment related to the different medical competency milestones. This allows other evaluators to answer the questionnaires related to the different medical competency milestones. To fulfill the residency requirements of their particular residency year, the medical resident will likely need to get a passing grade with respect to each question on each questionnaire related to each medical competency milestone. Therefore steps 206-214 can be repeated, as described above, until passing grades (i.e. an answer of “proficient”) is received in response to each relevant question. In some cases, a notification can be provided to the medical resident once a passing grade is received for each question on a given questionnaire. This signals to the medical resident that they have completed their medical residency requirements for that year-level in that particular competency area milestone. At that time, the medical resident need not submit any more solicitations to faculty for evaluation for that particular competency area milestone.

Alternatively, steps 206-214 can be repeated until the end of an evaluation period or until a certain given time period runs out. For example, if the medical resident is expecting a mid-year evaluation, steps 206-214 might be repeated for 6 months, until such a time as the residents progress is expected to be reviewed. At that point, the progress of the resident can be evaluated at step 216. During the evaluation process of step 216, the answers provided by the evaluators can be relied on to determine the progress of the medical resident. A report can also be generated, at step 218, to help show the current progress of the medical resident. For example, the report can show which medical competency milestones the medical resident has achieved a passing grade in (i.e. an answer indicating they are proficient in response to all related questions), and which medical competency milestone the resident must still achieve a passing grade in to complete the requirements for that year of their medical residency. A report can be generated efficiently since the questions and answers are streamlined in most cases, as described above. The report generated at step 218 can then be relied upon by a reviewer to quickly and efficiently provide a review of the medical resident's performance. This can be helpful in periodic reviews, such as during a semi-annual review of the resident's progress. Further, the resident may take the initiative to generate and/or use a progress report to keep track of their progress even absent input from a reviewer. In any case, once the resident has completed their medical residency for that year, an overall report is generated at step 220 to document the answers provided by evaluators with respect to each competency milestone area, thereby documenting the overall progress of the medical resident. Therefore, while evaluation is performed in a modular fashion, with data on specific competency areas gathered separately during different clinical interactions and potentially by different evaluators, this data can then be totaled to create an overall evaluation report.

While the process 200 involves e-mailing access codes to residents which the resident then provide to evaluators so the evaluator can access the questionnaire, it should be noted that in other embodiments, other methods are used. For example, in some embodiments, no e-mail or access code is sent to the medical resident. Rather, questionnaires are generated and once the evaluator works with the resident in a healthcare area related to one of the competency area milestones, the evaluator is provided with a questionnaire to fill out that is related to that competency area milestone. In other words, in some cases, the evaluator can gain access to, or be provided with, a relevant questionnaire without any interaction with the medical resident (or at least without receiving an e-mail). The evaluator can be provided with the questionnaire through a computer, a mobile application, by hand, or by any other method of delivery. Therefore implementation of the subject technology is not limited to the process 200.

The subject technology, as described above, reinforces the structure of the milestones with both residents and evaluators. In addition, the system fosters proactive initiative on the part of the resident in his or her own learning. The resident is tasked with the responsibility of soliciting these individual “mini-evaluations” from the faculty with which the resident worked, and incentivized to make these requests as closely in time as possible to when the interaction occurred for which the resident is requesting the assessment. The resident is therefore motivated to keep track of which specific milestones have and have not been solicited and evaluated, as the resident's training progresses.

It will be appreciated by those of ordinary skill in the pertinent art that the functions of several elements may, in alternative embodiments, be carried out by fewer elements, or a single element. All processes shown and described herein, in different embodiments, may be carried out by executing the steps in a different order, or by omitting a step or adding additional steps.

While the subject technology has been described with respect to preferred embodiments, those skilled in the art will readily appreciate that various changes and/or modifications can be made to the subject technology without departing from the spirit or scope of the subject technology. For example, each claim may depend from any or all claims in a multiple dependent manner even though such has not been originally claimed.

1. A process of evaluating the residential clinical performance of a medical resident based upon feedback from a plurality of evaluators, comprising:
(I) generating a plurality of questionnaires, each questionnaire: related to one of a plurality of competency area milestones; and having a plurality of questions related to the one of a plurality of competency area milestones;
(II) sending a plurality of e-mails to the medical resident, each e-mail containing an access code which provides access to one of the plurality of questionnaires;
(III) working, by the medical resident, with one of the plurality of evaluators on a patient receiving healthcare related to one of the competency area milestones;
(IV) sending, by the medical resident to the one of the evaluators, one of the access codes providing access to one of the plurality of questionnaires that relates to the one of the competency area milestones;
(IV) utilizing the access code, by the one of the evaluators, to access the one of the questionnaires;
(V) providing a plurality of answers, by the one of the evaluators, to one or more of the questions on the questionnaire that was accessed; and
(VI) logging the answers in an evaluation database.

2. The process of claim 1 wherein the competency area milestones are related to healthcare treatment areas in which the medical resident is being evaluated.

3. The process of claim 2 wherein the healthcare treatment areas are based on the years of medical residency completed by the medical resident.

4. The process of claim 1 wherein in step (V), the answers are provided via a mobile device through an application.

5. The process of claim 1 wherein each questionnaire contains five or fewer questions.

6. The process of claim 3 wherein in step (V) the answers are provided by selecting one of five options.

7. The process of claim 6 wherein the five options are the following: proficient;
competent; early learner; unsatisfactory; and pass.

8. The process of claim 7, further comprising the steps of:
(VII) repeating steps (III) through (VI) until “proficient” is selected at least once as an option in response to each question on one of the plurality questionnaires; and
(VIII) notifying the medical resident that “proficient” was selected at least once as an option in response to each question the one of the plurality of questionnaires.

9. The process of claim 7, further comprising the steps of:
(VII) repeating steps (III) through (VI) throughout an evaluation period; and
(VIII) determining the total progress made by the medical resident towards competency area milestones during the evaluation period, the total progress being based upon the answers provided by the evaluators.

10. The process of claim 7, further comprising:
(VII) creating an overall evaluation report based upon the answers provided.

11. The process of claim 8, further comprising:
(VI)(a) determining the progress of the medical resident towards their competency area milestones based upon the answers provided.

12. A process of evaluating the residential clinical performance of a medical resident based upon feedback from a plurality of evaluators, comprising:
(I) generating a plurality of questionnaires, each questionnaire: related to one of a plurality of competency area milestones; and having a plurality of questions related to the one of a plurality of competency area milestones;
(II) working, by the medical resident, with one of the plurality of evaluators on a patient receiving healthcare related to one of the competency area milestones;
(III) providing one of the questionnaires to the one of the plurality of evaluators, the one of the questionnaires being related to the one of the competency area milestones;
(IV) providing a plurality of answers, by the one of the evaluators, to one or more of the questions on the one of the questionnaires; and
(V) logging the answers in an evaluation database.

13. The process of claim 11 wherein in step (IV) the answers are provided by selecting one of five options.

14. The process of claim 12 wherein the five options are the following: proficient;
competent; early learner; unsatisfactory; and pass.

15. The process of claim 13 further comprising:
(VI) determining the progress of the medical resident towards their competency area milestones based upon the answers provided.

16. The process of claim 14 further comprising:
(VI) repeating steps (II) through (V) throughout an evaluation period; and
(VII) determining the total progress made by the medical resident towards competency area milestones during the evaluation period, the total progress being based upon the answers provided by the evaluators.

17. The process of claim 16, further comprising:
(VIII) creating an overall evaluation report based upon the answers provided.

18. The process of claim 17 wherein the overall evaluation report identifies competency area milestones in which, on the questionnaire related to that competency area milestone, at least one evaluator has provided an answer of “proficient” for each question.
